(19) United States
(12) Patent Application Publication
(10) Pub. No.: US 2022/0031880 A1
(43) Pub. Date: Feb. 3, 2022

(54) ULTRAVIOLET STERILIZATION APPARATUS FOR VEHICLE

(71) Applicant: KYUNGSHIN CO., LTD, Incheon (KR)

(72) Inventors: Chung Yul HWANG, Incheon (KR); Chang Suk KIM, Incheon (KR); Nak Sun SUNG, Incheon (KR); Yun Suk CHOI, Incheon (KR)

(21) Appl. No.: 17/334,781

(22) Filed: May 30, 2021

(30) Foreign Application Priority Data
Jul. 28, 2020 (KR) 10-2020-0093900

(51) Int. Cl. A61L 2/10; A61L 2/24; A61L 9/20

(52) U.S. Cl. CPC A61L 2/10; A61L 2/24; A61L 2202/25; A61L 2202/11; A61L 2202/14; A61L 9/20

(57) ABSTRACT

An ultraviolet (UV) sterilization apparatus includes a main power input unit to receive main power from a vehicle; an auxiliary power input unit to receive auxiliary power from an auxiliary battery; a main power detection unit to detect a state of main power input from the main power input unit; an auxiliary power detection unit to detect a charging state of the auxiliary battery; a vehicle information input unit to receive vehicle information for determining a state of the vehicle; a power conversion unit to switch between the main power and the auxiliary power input from the main power input unit and the auxiliary power input unit; a plurality of UV modules installed at a plurality of locations in the vehicle and configured to irradiate UV rays; an UV driving unit configured to drive the plurality of UV modules; and a control unit to operate features thereof.

### CROSS-REFERENCE TO RELATED APPLICATION

This application claims priority from and the benefit of Korean Patent Application No. 10-2020-0093900, filed on Jul. 28, 2020, which is hereby incorporated by reference for all purposes as if fully set forth herein.

### BACKGROUND

### Field

Exemplary embodiments relate to an ultraviolet sterilization apparatus for a vehicle.

### Discussion of the Background

In general, air conditioners for vehicles have the functions of constant temperature and constant humidity to keep constant temperature and constant humidity as well as functions such as dehumidification, humidification, purification, ventilation, aroma, in addition to cooling and heating, which are the main functions so as to maintain the indoor air environment of the vehicle in a comfortable or suitable condition for the driver.

In such air conditioners for vehicles, because air is introduced into the interior of the vehicle through a cabin filter that filters and purifies pollutants such as exhaust gas, dust, and yellow dust in the air, the cabin filter plays an important role in whether indoor air is comfortable or not.

However, because the cabin filter is usually located inside a glove box in front of a passenger seat, when the cabin filter is inspected and replaced, the glove box needs to be opened, separated, and then reinserted. Also, because the cabin filter needs to be mounted in a predetermined direction in which the air flows, i.e., according to directivity, it is very cumbersome and laborious for the driver to replace the cabin filter from time to time, and it is difficult to immediately check a contamination state, so it takes quite a long time, and it is not easy to carry out.

The background technology of the present invention is disclosed in Korean Patent Laid-open Publication No. 10-2020-0075412 (published on Jun. 26, 2020, Air Cleaner for Vehicle).

On the other hand, because the air contains various pollutants such as bacteria, viruses, mold, fine dust, harmful gases and odor components that are harmful to health, the demand for air purifiers for vehicles having an air purification function using a filter and a drying function using a blower so as to improve and maintain the indoor air quality of a vehicle has been increased.

Recently, air purifiers have been developed to control bacteria and purify indoor air by applying the sterilization effect of an ultraviolet (UV) light emitting diode (LED) that emits UV rays for air purification.

In particular, as large-scale infectious diseases (SARS/H1N1 flu/MERS/Corona 19, etc.) continue to occur, interest in sterilization and disinfection is also increasing, and the need for sterilization is increasing even in automobiles that are necessary for daily life.

The above information disclosed in this Background section is only for enhancement of understanding of the background of the invention and, therefore, it may contain information that does not constitute prior art.

### SUMMARY

Exemplary embodiments of the present invention provide an ultraviolet sterilization apparatus for a vehicle, in which a plurality of ultraviolet light emitting diodes (LEDs) installed inside the vehicle are independently driven to uniformly sterilize the interior space without a shaded area and the driving state of the vehicle and the state of a passenger are determined by receiving vehicle information so that the operation of the plurality of LEDs is controlled and discharge of the vehicle is prevented by utilizing an auxiliary battery.

According to an aspect of the present invention, there is provided an ultraviolet (UV) sterilization apparatus for a vehicle, including a main power input unit configured to receive main power from a vehicle, an auxiliary power input unit configured to receive auxiliary power from an auxiliary battery, a main power detection unit configured to detect a state of main power input from the main power input unit, an auxiliary power detection unit configured to detect a charging state of the auxiliary battery connected to the auxiliary power input unit, a vehicle information input unit configured to receive vehicle information for determining a state of the vehicle, a power conversion unit configured to switch between the main power and the auxiliary power input from the main power input unit and the auxiliary power input unit, respectively, a plurality of UV modules installed at a plurality of locations in the vehicle and configured to irradiate UV rays, an UV driving unit configured to drive the plurality of UV modules, and a control unit configured to receive the vehicle information input from the vehicle information input unit and the state of the main power and the charging state of the auxiliary battery, determine an operating condition of the UV modules, and control the power conversion unit so as to drive the plurality of UV modules independently through the UV driving unit during a sterilization driving time.

The UV sterilization apparatus for the vehicle may further include an operation display unit configured to display an operating state and result of the control unit.

The UV driving unit may include a plurality of ports which allow driving power to be output differently according to the UV modules.

The control unit may determine a case where an absence state of a passenger is maintained for more than a set time, as the operating condition based on any one or more of a starting state, a shifting state, a door opening/closing state, a door locking state, a seating state, an audio/video/navigation (AVN) state, an emergency light lighting state, a seat movement state, an fob signal state, and a rear seat detection state from the vehicle information.

When the control unit determines the operating condition and then a charging state of the main battery is less than expected driving power, the control unit may switch the power conversion unit to the auxiliary power so as to drive the UV driving unit.

When an abnormality occurs in the state of the main power while the UV driving unit is operating, the control unit may switch the power conversion unit to the auxiliary power so as to operate the UV driving unit.

When the control unit determines the operating condition and then a charging state of the main battery is less than a set value, the control unit may stop an operation of the UV driving unit.

When a door lock is released while the UV driving unit is operating, the control unit stops the operation of the UV driving unit and temporarily stops counting of the sterilization driving time, and when the operating condition is satisfied again, the control unit may count the sterilization driving time so as to operate the UV driving unit again.

When the control unit determines again whether the operating condition is satisfied and then the operating condition is not satisfied, the control unit may stop the operation of the UV driving unit and may initialize counting.

The sterilization driving time may be set based on any one or more of a starting time, a door opening time, and temperature and humidity inside the vehicle.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the principles of the invention.

FIG. 1 is a block diagram illustrating an ultraviolet sterilization apparatus for a vehicle according to an embodiment of the present invention;

FIG. 2 is a diagram illustrating installation positions of ultraviolet (UV) modules in the UV sterilization apparatus for a vehicle according to an embodiment of the present invention; and

FIG. 3 is a flowchart illustrating a method of controlling a UV sterilization apparatus for a vehicle according to an embodiment of the present invention.

### DETAILED DESCRIPTION OF THE ILLUSTRATED EMBODIMENTS

The invention is described more fully hereinafter with reference to the accompanying drawings, in which embodiments of the invention are illustrated. This invention may, however, be embodied in many different forms and should not be construed as limited to the embodiments set forth herein. Rather, these embodiments are provided so that this disclosure is thorough, and will fully convey the scope of the invention to those skilled in the art. In the drawings, the size and relative sizes of layers and regions may be exaggerated for clarity.

Like reference numerals in the drawings denote like elements.

As is traditional in the corresponding field, some exemplary embodiments may be illustrated in the drawings in terms of functional blocks, units, and/or modules. Those of ordinary skill in the art will appreciate that these block, units, and/or modules are physically implemented by electronic (or optical) circuits such as logic circuits, discrete components, processors, hard-wired circuits, memory elements, wiring connections, and the like. When the blocks, units, and/or modules are implemented by processors or similar hardware, they may be programmed and controlled using software (e.g., code) to perform various functions discussed herein. Alternatively, each block, unit, and/or module may be implemented by dedicated hardware or as a combination of dedicated hardware to perform some functions and a processor (e.g., one or more programmed processors and associated circuitry) to perform other functions. Each block, unit, and/or module of some exemplary embodiments may be physically separated into two or more interacting and discrete blocks, units, and/or modules without departing from the scope of the inventive concept. Further, blocks, units, and/or module of some exemplary embodiments may be physically combined into more complex blocks, units, and/or modules without departing from the scope of the inventive concept.

Unless particularly described to the contrary, the term “comprise”, “configure”, “have”, or the like, which are described herein, will be understood to imply the inclusion of the stated components, and therefore should be construed as including other components, and not the exclusion of any other elements.

Hereinafter, an ultraviolet sterilization apparatus for a vehicle according to the present invention will be described with reference to the accompanying drawings. In this process, the thicknesses of lines or the sizes of components illustrated in the drawings may be exaggerated for clarity and convenience of description. In addition, terms to be described below are terms defined in consideration of functions in the present invention, which may vary according to the intention or custom of users or operators. Therefore, definitions of these terms should be made based on the contents throughout the present specification.

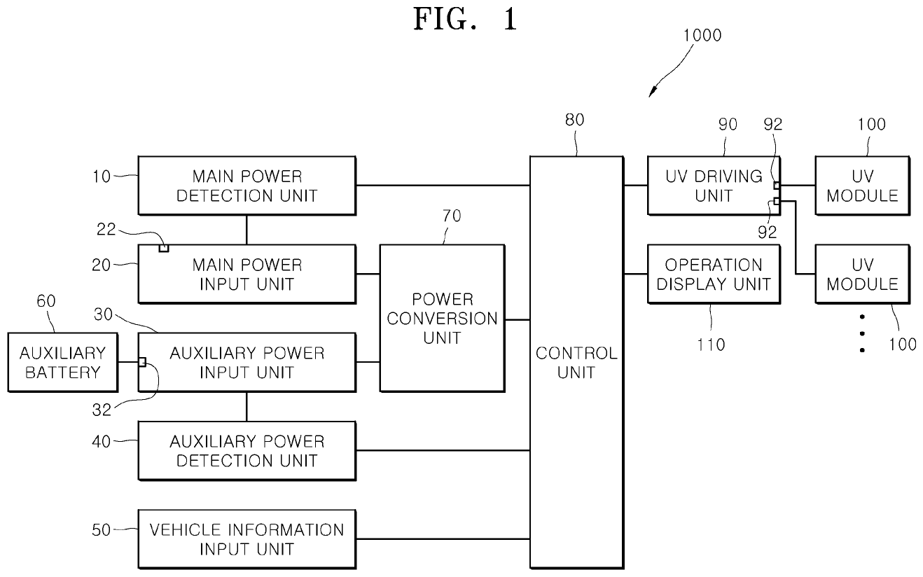

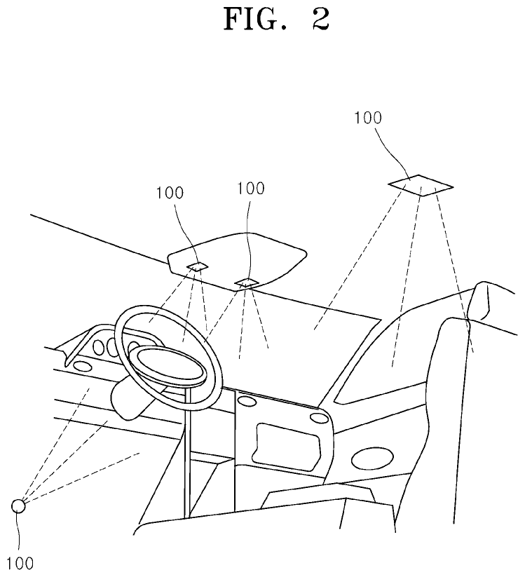

FIG. 1 is a block diagram illustrating an ultraviolet sterilization apparatus for a vehicle according to an embodiment of the present invention, and FIG. 2 is a view illustrating installation positions of ultraviolet (UV) modules in the UV sterilization apparatus for the vehicle according to an embodiment of the present invention.

As illustrated in FIG. 1, a UV sterilization apparatus 1000 according to the embodiment of the present invention may include a main power input unit 20, an auxiliary power input unit 30, a main power detection unit 10, an auxiliary power detection unit 40, a vehicle information input unit 50, a power conversion unit 70, a plurality of UV modules 100, an UV driving unit 90, and a control unit 80, as well as an operation display unit 110.

The main power input unit 20 includes at least one port 22 configured to receive main power supplied from a main battery of a vehicle.

The auxiliary power input unit 30 includes at least one port 32 configured to receive auxiliary power from an auxiliary battery 60.

The main power detection unit 10 may detect a state of main power input from the main power input unit 20.

The auxiliary power detection unit 40 may detect the charging state of the auxiliary battery 60 through the auxiliary power input unit 30.

The vehicle information input unit 50 may receive vehicle information to determine the state of the vehicle through vehicle internal communication. Information may be input to the vehicle information input unit 50 via various sensors dispersed throughout the vehicle.

Vehicle information may include at least one of a starting state, a shifting state, a door opening/closing state, a door locking state, a seating state, an audio/video/navigation (AVN) state, an emergency light lighting state, a seat movement state, a fob signal state, a rear seat detection state, and the charging state of the main battery.

The power conversion unit 70 is configured to switch between main power and auxiliary power input from the main power input unit 20 and the auxiliary power input unit 30, respectively, so that power can be stably supplied.

A plurality of UV modules 100 include UV light emitting didoes (LEDs), and are installed at a plurality of locations in the vehicle to irradiate UV rays throughout an interior of the vehicle and sterilize the interior of the vehicle.

Here, the plurality of UV modules 100 may be installed to irradiate UV rays to the interior of the vehicle at various locations. One location may be a location of a central interior light. Another location may be a front interior light of the ceiling of the vehicle. The combination of locations may ensure that UV rays can be irradiated throughout the vehicle without a shaded area inside the vehicle blocking UV rays, as illustrated in FIG. 2. The plurality of UV modules 100 may be installed to irradiate UV rays from different ceiling locations, from a lower space adjacent a driver's seat or passenger seat to the upper side and may be installed to irradiate UV rays from the left and right sides of the vehicle to all inside areas. UV modules 100 may be installed in the backseat areas of vehicles, and in way back areas that may be used for storage.

Other types of vehicles such as storage vans and passenger vans may have UV modules 100 and power units dispersed throughout to sterilize the indoor environment. Larger vehicles such as tractor trailers may be outfitted with the UV modules 100. Other passenger vehicles with closed spaces such as airplanes, buses, subway and train cars may also be fitted with UV modules 100 with accompanying power apparatuses and control units as described herein.

As illustrated in FIG. 1, the UV driving unit 90 may be provided with a plurality of ports 92 to independently drive the UV modules 100, so that the driving power can be output differently for different UV modules 100 or the driving can be performed for different times according to the installation location of the plurality of UV modules 100.

The control unit 80 is configured to receive vehicle information input from the vehicle information input unit 50, the state of main power, and the charging state. The control unit 80 determines the operating condition of the UV modules 100, and controls a power switching unit to drive the UV driving unit 90 during a sterilization driving time.

In accordance with embodiments described herein, the sterilization driving time may be set based on any one or more of a starting time of the vehicle, a door opening time of any one of the doors, and temperature and humidity measurements, odor, or other environmental factors inside the vehicle.

The control unit 80 may determine a case where an absent state of the passenger is maintained for more than a set time in conjunction with other sensed vehicle characteristics such as an operating condition based on a starting state, the shifting state, the door opening/closing state, the door locking state, the seating state, the audio, video, navigation (AVN) state, the emergency light lighting state, the seat movement state, the fob signal state and the rear seat detection state from the vehicle information. Depending on a combination of factors, the control unit 80 may control the various UV modules 100 to drive at various times and for various durations.

For example, when a user first presses a fob to unlock a car door, the system 1000 may be programmed to turn on one or more UV modules 100 within a predetermined time after receiving the press. The system may have a set initial period to turn on in anticipation of a user entering the vehicle. If the initial time period passes and no user enters the vehicle, the UV modules 100 may be switched off. If a user enters the vehicle within the initial time period, the UV modules 100 will stay on until programmed to turn off again.

In this way, the control unit 80 may determine the absence state of the driver by determining whether an engine is turned off, whether the vehicle is switched to a parking mode, whether a door is closed, or whether the door is locked and no weight is detected on the seat from the vehicle information. In addition, the control unit 80 determines the absence state of the passenger by determining whether the AVN state is an activated state, emergency lights are turned on, seats are moving, fob signals are detected inside the vehicle, and a rear seat detection state is input, and the control unit 80 is configured to determine the case where the absence state of the passenger is maintained for more than a set time, as an operating condition, so that the safety of the passenger due to UV sterilization can be considered as much as possible.

Thereafter, when the state of the main power is normal after the control unit 80 determines the operating condition, the control unit 80 switches the power conversion unit 70 to the main power so as to operate the UV driving unit 90. Alternatively, the control unit 80 determines the charging state of the auxiliary battery 60, and when the charging state of the auxiliary battery 60 is greater than or equal to predicted driving power, the control unit 80 switches the power conversion unit 70 to the main power so as to operate the UV driving unit 90. By doing so, the control unit 80 may determine the secured state of the driving power and then stably operate the sterilization apparatus even when an abnormality of the main power occurs during the operation of the sterilization apparatus.

In addition, the control unit 80 determines the operating condition, and when the charging state of the main battery is less than the expected driving power, or when an abnormality occurs in the state of the main power while the UV driving unit 90 is operating, the control unit 80 switches the power conversion unit 70 to auxiliary power so as to operate the UV driving unit 90, thereby stably operating the operation of the sterilization apparatus.

Meanwhile, when the control unit 80 determines the operating condition and then the charging state of the main battery is less than a set value, the control unit 80 may stop the operation so as not to affect the operation of the vehicle.

In addition, when the door lock is released while the UV driving unit 90 is operating, the control unit 80 stops the operation of the UV driving unit 90 for safety and temporarily stops counting of the sterilization driving time, and when the operating condition is satisfied again, the control unit 80 counts the sterilization driving time so as to operate the UV driving unit 90 again, so that power consumption due to repeated sterilization can be reduced.

On the other hand, the control unit 80 temporarily stops the UV driving unit 90 and then determines whether the operation condition is satisfied again, and initializes the counting when the operating condition is not satisfied.

The operation display unit 110 displays the operating state and result of the control unit 80 so that the driver can visually check the operating state or operation result of the sterilization apparatus.

For example, the operation or completion state can be indicated through the color of an LED lamp, the progress state or progress result can be displayed sequentially by a plurality of LED lamps, and the operating state of the sterilization apparatus can be visually displayed by scanning a laser beam.

As described above, according to the UV sterilization apparatus for a vehicle according to the embodiment of the present invention, a plurality of UV LEDs installed inside the vehicle are independently driven to uniformly sterilize the interior space without a shaded area. The driving state of the vehicle and the state of the passenger(s) are determined by receiving vehicle information so that the operation of the plurality of LEDs is controlled and discharge of the vehicle is prevented by utilizing an auxiliary battery.

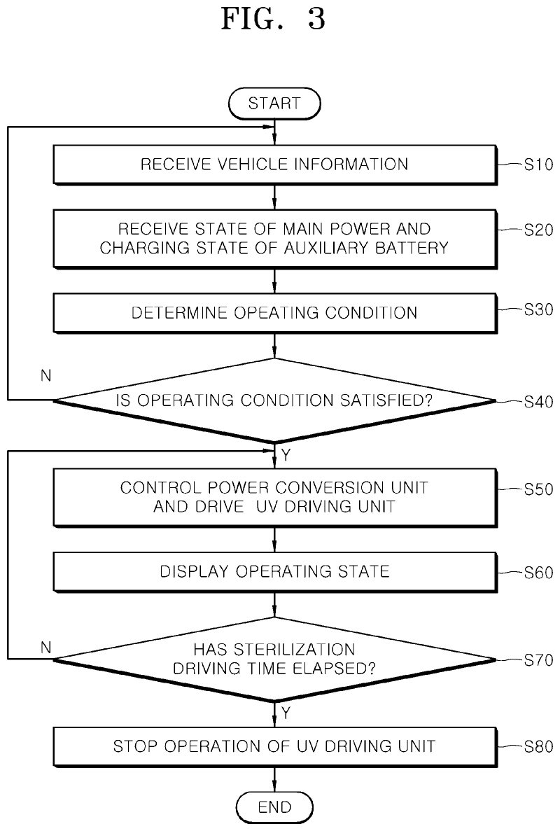

FIG. 3 is a flowchart illustrating a method of controlling a UV sterilizing apparatus for a vehicle according to an embodiment of the present invention.

As illustrated in FIG. 3, in the method of controlling a UV sterilization apparatus 1000 for a vehicle according to the embodiment of the present invention, first, the control unit 80 receives vehicle information from various vehicle sensors to determine the state of the vehicle from the vehicle information input unit 50 (S10).

In addition, the control unit 80 receives the state of the main power and the charging state of the auxiliary battery 60 from the main power detection unit 10 and the auxiliary power detection unit 40, respectively (S20).

After receiving the vehicle information, the state of the main power, and the charging state of the auxiliary battery 60 through operations S10 and S20, the control unit 80 determines the operating condition of the UV modules 100 based on the input vehicle information, the state of the main power, and the charging state of the auxiliary battery 60.

That is, the control unit 80 may determine a case where the absence state of one or more passengers is maintained for more than a set time. This absence may be an initial operating condition and be configured to relate to a starting state, a shifting state, a door opening/closing state, a door locking state, a seating state, an AVN state, an emergency light lighting state, a seat movement state, an fob signal state and a rear seat detection state from the vehicle information.

In this way, the control unit 80 may determine the absence state of a driver by determining whether an engine is turned off, is switched to a parking mode, a door is closed, the door is locked and no weight is detected on the seat from the vehicle information. In addition, the control unit 80 may determine the absence state of one or more passengers by determining whether, additionally, the AVN state is an activated state, emergency lights are turned on, seats are moving, fob signals are detected inside the vehicle, and a rear seat detection state is input. The control unit 80 may determine the case where the absence state of the passenger is maintained for more than a set time, as an operating condition, so that the safety of the passenger due to UV sterilization can be considered as much as possible.

After the operating condition of the UV modules 100 is determined in operation S30, the control unit 80 determines whether the operating condition is satisfied (S40).

When it is determined that the operating condition is not satisfied in operation S40 of determining whether the operating condition is satisfied, the control unit 80 returns to operation S10 and may repeatedly determine the operating condition of the UV modules 100 by receiving the state of main power and the charging state of the auxiliary battery 60. In this case, the above process may be repeated only for a predetermined time after the engine is turned off so as to prevent power consumption.

When it is determined that the operating condition is satisfied in operation S40 of determining whether the operating condition is satisfied, the control unit 80 controls the power conversion unit 70 so as to operate the UV driving unit 90 during a sterilization driving time (S50).

Here, when operating the UV driving unit 90, the control unit 80 may respectively adjust the operating time and operating strength of the UV modules 100 according to installation locations of the plurality of UV modules 100.

For example, when the vehicle information input unit 50 sends sensor data to the control unit that only a driver is present in the vehicle, the control unit 80 may instruct the UV driving unit 90 to drive the UV modules 100 at the front of a vehicle at full power. At substantially the same time the control unit 80 may instruct the UV driving unit 90 to drive the UV modules 100 in the backseat area and/or the storage area to operate at half power, twenty-five percent power, or the like. In this way power may be conserved in the system while providing sterilized air in the vicinity of the driver.

Also, when the state of the main power is normal, the control unit 80 switches the power conversion unit 70 to the main power so as to operate the UV driving unit 90, or determines the charging state of the auxiliary battery 60, and when the charging state of the auxiliary battery 60 is greater than or equal to the expected driving power, the control unit 80 switches the power conversion unit 70 to the main power so as to operate the UV driving unit 90, thereby determining the secured state of the driving power and then stably operating the sterilization apparatus even when an abnormality of the main power occurs during the operation of the sterilization apparatus.

In addition, when the charging state of the main battery is less than the expected driving power, or when an abnormality occurs in the state of main power while the UV driving unit 90 is operating, the control unit 80 switches the power conversion unit 70 to auxiliary power so as to operate the UV driving unit 90, thereby stably operating the operation of the sterilization apparatus.

Meanwhile, when the charging state of the main battery is less than a set value, the control unit 80 may stop the operation so as not to affect the operation of the vehicle.

In this case, the control unit 80 displays the operating state and result through the operation display unit 110 so that the driver can visually check the operating state or operation result of the UV driving unit 90 (S60).

For example, the operation or completion state can be indicated through the color of an LED lamp, the progress state or progress result can be displayed sequentially by a plurality of LED lamps, and the operating state of the UV driving unit 90 can be visually displayed by scanning a laser beam.

In this way, after operating the UV driving unit 90, the control unit 80 counts a sterilization driving time to determine whether the sterilization driving time has elapsed (S70).

When it is determined that the sterilization driving time has elapsed in operation S70 of determining whether the sterilization driving time has elapsed, the method returns to operation S50, and the UV driving unit 90 is operated during the sterilization driving time so that sterilization of the inside of the vehicle can be performed.

Here, the sterilization driving time may be set based on any one or more of a starting time, a door opening time, temperature and humidity measurements, odor, or other environmental factors inside the vehicle.

On the other hand, when it is determined that the sterilization driving time has elapsed in operation S70 of determining whether the sterilization driving time has elapsed, the control unit 80 stops the operation of the UV driving unit 90, initializes the counting and terminates the operation of the UV driving unit 90 (S80).

Meanwhile, when a door lock is released while the UV driving unit 90 is operating, the control unit 80 stops the operation of the UV driving unit 90 for safety and temporarily stops counting of the sterilization driving time, and when the operating condition is satisfied again, the sterilization driving time is counted and the UV driving unit 90 is operated again, so that power consumption due to repeated sterilization can be reduced.

On the other hand, after temporarily stopping counting of the sterilization driving time, the control unit 80 determines whether the operation condition is satisfied again, and when the operation condition is not satisfied, the control unit 80 initializes counting of the sterilization driving time and then terminates the operation of the UV driving unit 90.

As described above, according to the method of controlling a UV sterilization device for a vehicle according to the embodiment of the present invention, a plurality of UV light emitting diodes (LEDs) installed inside the vehicle are independently driven to uniformly sterilized the interior space without a shaded area and the driving state of the vehicle, and the state of a passenger are determined by receiving vehicle information so that the operation of the plurality of LEDs is controlled and discharge of the vehicle is prevented by utilizing an auxiliary battery.

The implementations described herein may be implemented in, for example, a method or process, an apparatus, a software program, a data stream or a signal. Although discussed in the context of a single form of implementation (e.g., as a method), the implementation of the discussed features may also be implemented in other forms (e.g., an apparatus or program). The device may be implemented with appropriate hardware, software and firmware. The method may be implemented in an apparatus such as a processor, which generally refers to a processing device including, for example, a computer, a microprocessor, an integrated circuit or a programmable logic device, or the like. Processors also include communication devices such as computers, cell phones, personal digital assistants (“PDAs”) and other devices that facilitate communication of information between end-users.

In the UV sterilization apparatus for a vehicle according to the aspect of the present invention, a plurality of LEDs installed inside the vehicle are independently driven so that the interior space can be uniformly sterilized without a shaded area and the driving state of the vehicle, and the state of a passenger are determined by receiving vehicle information so that the operation of the plurality of LEDs is controlled and discharge of the vehicle is prevented by utilizing an auxiliary battery.

While one or more embodiments have been described with reference to the figures, it will be understood by those of ordinary skill in the art that various changes in form and details may be made therein without departing from the spirit and scope as defined by the following claims.

What is claimed is:

1. An ultraviolet (UV) sterilization apparatus for a vehicle, comprising:
a main power input unit configured to receive main power from a vehicle;
an auxiliary power input unit configured to receive auxiliary power from an auxiliary battery;
a main power detection unit configured to detect a state of main power input from the main power input unit;
an auxiliary power detection unit configured to detect a charging state of the auxiliary battery connected to the auxiliary power input unit;
a vehicle information input unit configured to receive vehicle information for determining a state of the vehicle;
a power conversion unit configured to switch between the main power and the auxiliary power input from the main power input unit and the auxiliary power input unit, respectively;
a plurality of UV modules installed at a plurality of locations in the vehicle and configured to irradiate UV rays;
is an UV driving unit configured to drive the plurality of UV modules; and
a control unit configured to receive the vehicle information input from the vehicle information input unit and the state of the main power and the charging state of the auxiliary battery, determine an operating condition of the UV modules, and control the power conversion unit so as to drive the plurality of UV modules independently through the UV driving unit during a sterilization driving time.

2. The UV sterilization apparatus for the vehicle of claim 1, further comprising an operation display unit configured to display an operating state and result of the control unit.

3. The UV sterilization apparatus for the vehicle of claim 1, wherein the UV driving unit comprises a plurality of ports which allow driving power to be output differently according to the UV modules.

4. The UV sterilization apparatus for the vehicle of claim 1, wherein the control unit determines a case where an absence state of a passenger is maintained for more than a set time, as the operating condition based on any one or more of a starting state, a shifting state, a door opening/closing state, a door locking state, a seating state, an audio/video/navigation (AVN) state, an emergency light lighting state, a seat movement state, an fob signal state, and a rear seat detection state from the vehicle information.

5. The UV sterilization apparatus for the vehicle of claim 1, wherein, when the control unit determines the operating condition and then a charging state of the main battery is less than expected driving power, the control unit switches the power conversion unit to the auxiliary power so as to drive the UV driving unit.

6. The UV sterilization apparatus for the vehicle of claim 1, wherein, when an abnormality occurs in the state of the main power while the UV driving unit is operating, the control unit switches the power conversion unit to the auxiliary power so as to operate the UV driving unit.

7. The UV sterilization apparatus for the vehicle of claim 1, wherein, when the control unit determines the operating condition and then a charging state of the main battery is less than a set value, the control unit stops an operation of the UV driving unit.

8. The UV sterilization apparatus for the vehicle of claim 1, wherein, when a door lock is released while the UV driving unit is operating, the control unit stops the operation of the UV driving unit and temporarily stops counting of the sterilization driving time, and when the operating condition is satisfied again, the control unit counts the sterilization driving time so as to operate the UV driving unit again.

9. The UV sterilization apparatus for the vehicle of claim 8, wherein, when the control unit determines again whether the operating condition is satisfied and then the operating condition is not satisfied, the control unit stops the operation of the UV driving unit and initializes counting.

10. The UV sterilization apparatus for the vehicle of claim 1, wherein the sterilization driving time is set based on any one or more of a starting time, a door opening time, and temperature and humidity inside the vehicle.
